(19) United States
(12) Patent Application Publication
(10) Pub. No.: US 2005/0177025 A1
(43) Pub. Date: Aug. 11, 2005

(54) PROPHYLACTIC POLYMER PROBE COVER

(76) Inventors: Marc Jaker, New Brighton, MN (US); Mark Nelson, St. Paul, MN (US)

Correspondence Address: SNELL & WILMER;ONE ARIZONA CENTER, 400 EAST VAN BUREN, PHOENIX, AZ, 850040001, US

(21) Appl. No.: 10/905,996

(22) Filed: Jan. 28, 2005

Related U.S. Application Data
(60) Provisional application No. 60/481,962, filed on Jan. 28, 2004.

(51) Int. Cl. A61B 1/04

(52) U.S. Cl. 600/121

(57) ABSTRACT

A closed end polymerric cover for medical probes such as pressure sensing urodynamic catheters, gastroesophogeal manometer probes, televideo probes, such as endoscopes, of which there are several types, and the like. The polymeric covers will minimize the cleaning of the proboscopic devices between procedures/patients. Thus, the closed end polymeric probe is more aseptic and cost effective to the current required FDA protocols and also reduce the number of probes a clinic/hospital may need to possess. The distal end may be a flexible, perhaps clarified, clear window to permit the endoscope to survey the body cavity. The sheathing material in one embodiment is preferably a chemically bio-inert Fluoropolymer such as polytetrafluoroethylene (PTFE), fluorinate ethylene propylene (FEP), perfluoroalkoxy (PFA), or polyethylene terephthalate (PET), Urethane, Silicone, polyethylene, or another similar singular high slip substrate film that facilitates covering and uncovering the probe.

### CROSS-REFERENCE TO RELATED APPLICATIONS

This application claims priority to, and the benefit of, U.S. Provisional Patent Application Ser. No.: 60/481,962, entitled “PROPHYLACTIC POLYMER PROBE COVER” and filed Jan. 28, 2004.

### FIELD OF INVENTION

The present invention relates generally to medical diagnostic probes and vision endoscopes used in minimally invasive surgical procedures, diagnostics and medical examinations. The cleaning of these medical tools typically utilizes caustic chemicals that are regulated by the Environmental Protection Agency (EPA) and the Occupational Health and Safety Administration (OSHA). These EPA and OSHA regulated and Food and Drug Administration (FDA) protocol mandated chemicals degrade these medical tools, thus shortening their useable life.

In particular, the present invention relates to polymeric probe covers that can minimize or eliminate the need for caustic cleaning chemicals. Additionally, a side “saddle bag(s)” access channel(s) may be offered for those procedures that also involve the need for a water/air line, a biopsy instrument, and the like. The present sheath offers a biobarrier between the probe, scope, or instrument and the patient.

### BACKGROUND OF INVENTION

Probes and scopes have been integral to medicine for many years. However, probes have fallen under increased scrutiny from physicians, hospitals, and the FDA for possible cross-contamination between patients. Since these probes are used in natural and surgically created body cavities, there is a risk that the lack of cleanliness and/or “sterility” will lead to a healthy patient being infected with viruses and bacteria such as Polio, Hepatitis, Herpes, HIV, Tuberculosis, and the like. Another risk is that a patient with one malady may be introduced to new, additional problems.

Most probes and scopes do not use a barrier and therefore, are governed by FDA, OSHA, and EPA protocols for cleaning with caustic germicides. These hazardous chemicals take up labor resources and time, and expose the medical staff to danger. In addition, the EPA and OSHA regulated and FDA protocol mandated chemicals degrade these medical tools, thus shortening their useable life. Some probes are much more difficult than others to clean as they include long, small channels used for biopsy instruments, water irrigation, air, suction, and the like, so flushing may not suffice. Scrubbing may also be necessary. Damage complicated by this additional handling and degradation of exposed surfaces can and does occur to these probes periodically adding to the overall costs of health care. Back up scopes are then required, in addition to this special cleaning lab.

### SUMMARY OF INVENTION

In one embodiment, the present invention utilizes a Fluoropolymer or similarly slippery, soft, flexible, non-elastic, thin film with side weld seams. The film may comprise Generation II polytetrafluoroethylene (PTFE) or a similarly performing polymer, such as perfluoroalkoxy (PFA) or fluorinate ethylene propylene (FEP) which has been tensilized for added slip and softness throughout the length of the main body. The film may have a seam at the tip to facilitate a probe with side pressure transducers or the film may have no seam at the tip, so as to avoid any visual obstruction, thus allowing for an integral clear window, such as, for vision Endoscope systems. Layers of film can be welded together longitudinally so as to create side pockets. All may be mounted on a deployment tube for ease of loading/unloading onto the probe.

### BRIEF DESCRIPTION OF THE DRAWINGS

A more complete understanding of the present invention may be derived by referring to the detailed description when considered in connection with the Figures, wherein like reference numbers refer to similar elements throughout the Figures, and:

FIG. 1 is a perspective view of polymer probe cover components in accordance with one embodiment of the present invention;

FIG. 2 is a perspective view of various aspects of polymer probe covers in accordance with various embodiments of the present invention;

FIG. 3 is a perspective view of various aspects of polymer probe covers in accordance with various embodiments of the present invention;

FIG. 4 is a perspective view of a polymer probe cover in accordance with one embodiment of the present invention;

FIG. 5 is a perspective view of polymer probe cover components in accordance with one embodiment of the present invention;

FIG. 6 is a perspective view of polymer probe cover components in accordance with one embodiment of the present invention;

FIG. 7 is a side view of polymer probe cover components in accordance with one embodiment of the present invention;

FIG. 8 is a side view of polymer probe cover components in accordance with one embodiment of the present invention;

FIG. 9 is a side view of polymer probe cover components in accordance with one embodiment of the present invention;

FIG. 10 is a side view of polymer probe cover components in accordance with one embodiment of the present invention;

FIG. 11 is a perspective view of polymer probe cover components in accordance with one embodiment of the present invention;

FIG. 12 is a perspective view of polymer probe cover components in accordance with one embodiment of the present invention; and

FIG. 13 is a side view of polymer probe cover components in accordance with one embodiment of the present invention.

### DETAILED DESCRIPTION

The present invention may be described herein in terms of various hardware components and modules and processing steps. It should be appreciated that such modules and steps may be realized by any number of hardware components configured to perform the specified functions. For example, the present invention may employ various shaped tubes, sheaths, and the like, which may carry out a variety of functions. In addition, those skilled in the art will appreciate that the present invention may be practiced in any number of contexts and that the illustrative embodiment as described herein is merely one exemplary application for the invention. For example, the present invention may be applicable to various types of animals or for other applications that require minimally invasive surgical procedures. Further, such general techniques that may be known to those skilled in the art are not described in detail herein.

The present invention minimizes or eliminates the need to use caustic chemicals to clean medical diagnostic probes and vision endoscopes that are used in minimally invasive surgical procedures, diagnostics and medical examinations. In addition, the polymer probe cover of the present invention provide for side “saddle bag” access channels that may be used for procedure that involve the need for a water/air line or a biopsy instrument and the like. It should be appreciated that the present invention provides for a polymer probe cover that acts as a biobarrier between the probe/scope and the patient.

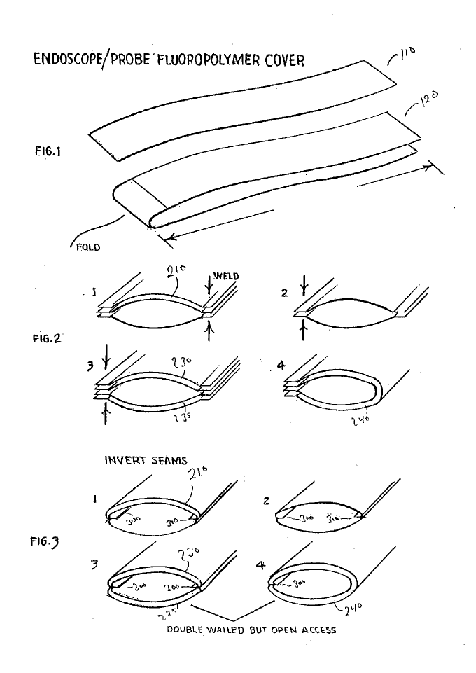

With reference to FIG. 1, in accordance with one aspect of the present invention, FIG. 1 illustrates two sheet of film 110 and 120 that form a membrane sheath. As illustrated, one sheet of film 120 is folded and the other sheet of film 110 may be layered into the folded sheet 120. Film 110, 120 comprise a thin, flexible polymeric substrate such as polytetrafluoroethylene (PTFE) or a similarly performing polymer, such as perfluoroalkoxy (PFA) or fluorinate ethylene propylene (FEP) which has been tensilized for added slip and softness throughout the length of the main body. The film may have a seam at the tip to facilitate a probe with side pressure transducers or the film may have no seam at the tip, so as to avoid any visual obstruction, thus allowing for an integral clear window, such as, for vision Endoscope systems. Layers of film can be welded together longitudinally so as to create side pockets.

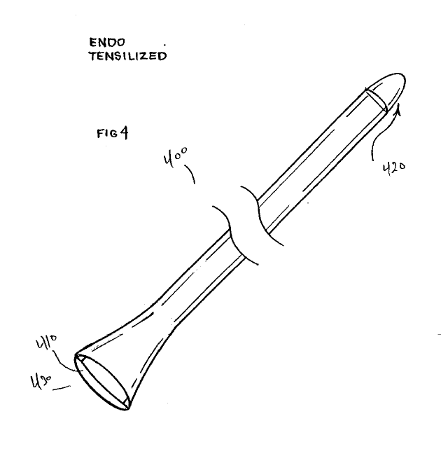

In accordance with another aspect of the present invention, FIG. 2 illustrates four embodiments of a probe cover that may be formed from layers of film 110, 120. Simple sheath 2 has zero side pockets. Film layers 110, 120 may be heat welded longitudinally so as to create side pockets. Seams 300 (for example, see FIG. 3) show where the film layers are heat welded. Simple sheath 1 includes one side pocket 210. Simple sheath 3 includes two side pockets 230, 235 and simple sheath 4 includes a tube within a tube offering nearly a 360 degree outer/side pocket 240. With reference to FIG. 3, the four embodiments of the sheath are illustrated with the seams 300 inverted to the inside. It should be appreciated that putting the seams to the inside helps with patient comfort.

With reference to FIG. 4, a sheath 400 is illustrated with an exemplary side pocket 410, and a distal 420 and proximal 430 end. The distal end 430 is clear and closed. The proximal end 430 is open which may then be loaded onto and affixed to a deployment tube.

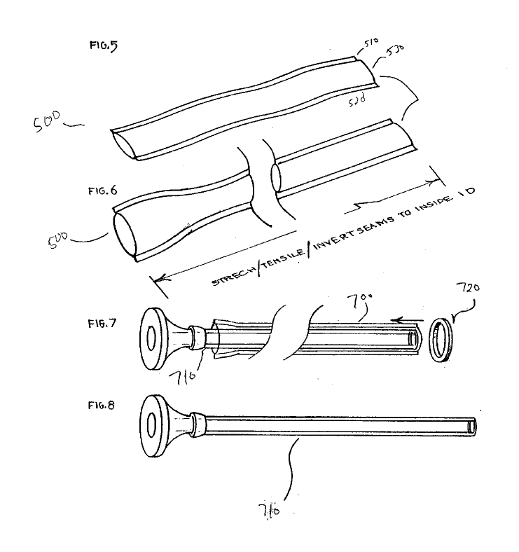

In accordance with another aspect of the present invention, FIG. 5 illustrates a sheath 500 with two seals 510, 520 on each side and a fold 530 at the distal end where the tip of the probe will locate. With reference to FIG. 6, the sheath 500 is stretched in its main body to improve the bio-barrier, and to increase slip, pliability, and softness.

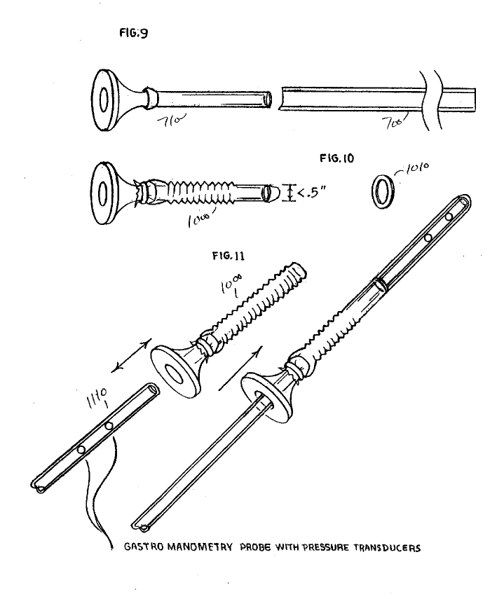

In accordance with another aspect of the present invention, FIG. 7 illustrates a sheath 700 being loaded onto a deployment tube 710. Once the sheath is in place near the proximal end, a snap ring 720 is positioned so that it is affixed to the sheath. With reference to FIG. 8, the deployment tube 700 is illustrated. In one embodiment, deployment tube 700 may comprise an injection molded deployment tube. The tube may be made of acrylic, styrene, polycarbonate, or similar material.

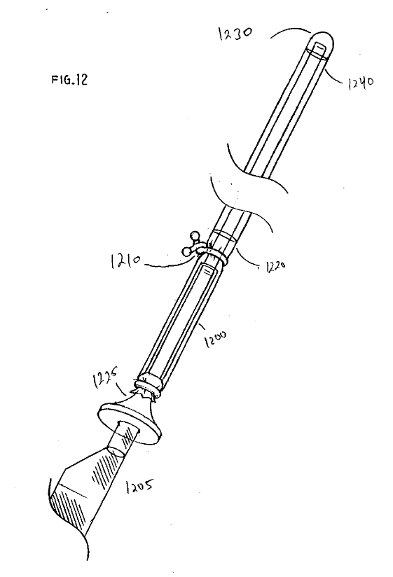

In accordance with another aspect of the present invention, FIG. 9 illustrates sheath 700 and deployment tube 710 having an approximate range of product length so as to accommodate various pressure, temperature, probes, and endoscopic systems and combined assemblages. With reference to FIG. 10, in accordance with one embodiment of the present invention, the assembly 1110 and estimated appropriate size for an ENT GI scope may be approximately 4-5 inches. A twist ring 1010 may be used for anchoring the sheath onto the scope to hold the sheath taut against the viewing window in its loaded position.

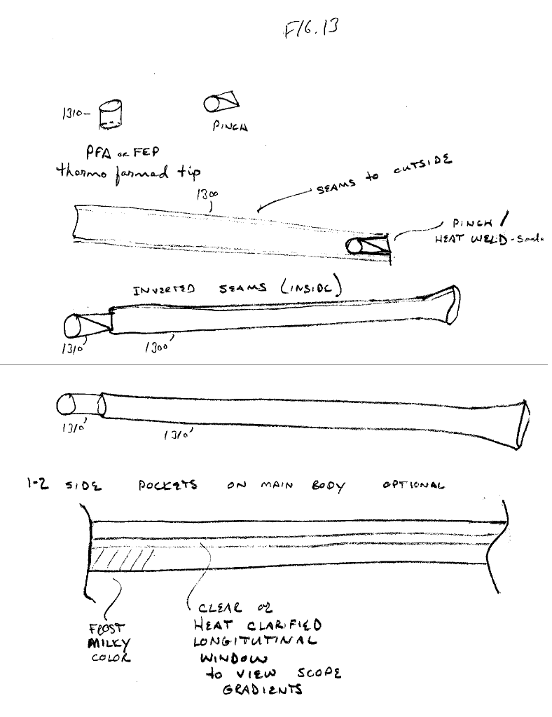

In accordance with another aspect of the present invention, FIG. 11 shows the assembly 1000 ready to load onto a probe 1110 and illustrates how the accordianed PTFE (or similarly performing polymer) unfolds as the probe is pushed against the distal end and through the deployment tube in the loading operation.

FIG. 12 illustrates a loaded sheath 1200 positioned on a vision scope 1205. A twist lock 1210 may be positioned over the flexible sheath 1200 in front of the rigid deployment tube 1225, but behind the entry 1220 to the side pocket. In addition, a clear tip 1230 and an exit 1240 to the side pocket are located at one end of sheath 1200.

With reference to FIG. 13, a sheath 1300 is illustrated with a separate thermo formed tip 1310. Tip 1310 may be heat sealed onto sheath 1300 such that a probe may be inserted into sheath 1300 with the optic end of the probe positioned proximate to tip 1310.

The present invention has been described above with reference to an exemplary embodiment. However, those skilled in the art will recognize that changes and modifications may be made to the exemplary embodiment without departing from the scope of the present invention. For example, the various processing steps dictated by the present invention, as well as the components for carrying out the processing steps, may be implemented in alternate ways depending upon the particular application or in consideration of any number of cost functions associated with the operation of the system. These and other changes or modifications are intended to be included within the scope of the present invention.

1. A probe cover assembly for covering a medical probe, the assembly comprising:
a first membrane sheath having a first end and a second end;
a second membrane sheath having a first end and a second end, wherein the first membrane sheath is heat welded to the second membrane sheath in a longitudinal fashion to create a left and right side pocket; and wherein the first ends of the first and second membrane sheaths comprises a clear window.

2. The probe cover assembly of claim 1, wherein the first and second membrane sheath comprises a fluoropolymer material.

3. The probe cover assembly of claim 1, wherein the first and second membrane sheath comprises a polyethylene material.

4. The probe cover assembly of claim 1 further comprising a deployment tube, such that the probe cover assembly is mounted on the deployment tube.
